(19) United States
(12) Patent Application Publication
(10) Pub. No.: US 2023/0391803 A1
(43) Pub. Date: Dec. 7, 2023

(54) COMPOSITIONS AND RELATED METHODS OF ALKYLTINTRIHALIDES

(71) Applicant: ENTEGRIS, INC., Billerica, MA (US)

(72) Inventors: David M. Ermert, Danbury, CT (US); Claudia Fafard, New Milford, CT (US); Thomas Coyne, Stormville, NY (US); Thomas M. Cameron, Newtown, CT (US)

(21) Appl. No.: 18/204,210

(22) Filed: May 31, 2023

Related U.S. Application Data
(60) Provisional application No. 63/400,269, filed on Aug. 23, 2022.
(60) Provisional application No. 63/348,859, filed on Jun. 3, 2022.

(51) Int. Cl. C07F 7/22; C07D 307/06; C07C 43/04

(52) U.S. Cl. CPC C07F 7/2208; C07D 307/06; C07C 43/043; C07F 7/2284

(57) ABSTRACT

The present disclosure includes a method of obtaining an alkyltintrihalide, obtaining a solvent, and contacting the alkyltintrihalide and the solvent, thereby forming an alkyltintrihalide adduct. Also described is a composition including: an alkyltintrihalide adduct of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

### PRIORITY

The present disclosure claims priority to U.S. provisional patent No. 63/348,859 with a filing date of Jun. 3, 2022 and U.S. provisional patent No. 63/400,269 with a filing date of Aug. 23, 2022. Both priority documents are incorporated by reference herein.

### BACKGROUND

### Field

The present disclosure relates to the field of compositions and related methods of alkyltintrihalides.

Films can be used in applications during the manufacture of microelectronic devices. Some of the films can be made using chemical vapor deposition or atomic layer deposition.

### SUMMARY

The present disclosure is directed to adducts of alkyltintrihalides, including solvent-based adducts of alkyltintrihalides, for production of high-purity (e.g., greater than 95% purity) extreme ultraviolet atomic layer deposition precursors. In some embodiments, the purity is greater than 99.8%. The present disclosure also relates to the synthesis of compounds, such as tris(dimethylamido) tin alkyl compounds, with low to no dialkyl impurities.

The present disclosure addresses the problem of producing high-purity tris(dimethylamido) alkyl tin compounds with low to no amount of dialkyl impurities. In addition, the present disclosure requires few steps to synthesize the compounds and low costs to produce the compounds.

The present disclosure describes direct routes to make RSn(NMe₂)₃. In some embodiments and as described further herein, R can be isopropenyl, isopropyl, or ethyl. A solvent-based approach allows the present disclosure to avoid the need for making HNMe₂ adducts of RSnCl₃ compounds to lower the R₂Sn(NMe₂)₂ impurity profile.

The syntheses are R-group and solvent/solution dependent. Solvent/solution selection (e.g., tetrahydrofuran (THF), dimethoxyethane (DME), or hexanes) are a factor in providing adequate yields and purity of the final product.

The present disclosure uses coordinating solvents to form isolable alkyltintricholoride adducts alkylSnCl₃(solv)₁₋₂, which effectively provide a synthon for producing the desired tris(dimethylamido)alkyl tin products of interest in high-purity. The methods of the present disclosure require fewer reagents and steps than the current processes to manufacture alkyltintricholoride adducts and therefore presents a faster and less expensive process.

In some aspects, the techniques described herein relate to a method including: obtaining an alkyltintrihalide; obtaining a solvent, a solution, or any combination thereof; and contacting the alkyltintrihalide with the solvent, the solution, or any combination thereof so as to form an alkyltintrihalide adduct.

In some aspects, the techniques described herein relate to a method, wherein the method does not include forming an alkyltintrihalide-amine adduct.

In some aspects, the techniques described herein relate to a method, wherein the alkyltintrihalide-amine adduct is an alkyltintrihalide-(HNMe₂) adduct.

In some aspects, the techniques described herein relate to a method, wherein the alkyltintrihalide is a compound of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

In some aspects, the techniques described herein relate to a method, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

In some aspects, the techniques described herein relate to a method, wherein R is a vinyl, an allyl, a propynyl, a propenyl, or any isomer thereof.

In some aspects, the techniques described herein relate to a method, wherein the solvent includes at least one of tetrahydrofuran (THF), dimethoxyethane (DME), or any combination thereof.

In some aspects, the techniques described herein relate to a method, wherein the solution includes at least one of hexane, pentane, toluene, or any combination thereof.

In some aspects, the techniques described herein relate to a method, wherein the solvent includes at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

In some aspects, the techniques described herein relate to a method, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

In some aspects, the techniques described herein relate to a method including: obtaining an alkyltintrihalide adduct; obtaining a lithium dialkylamide; and contacting the alkyltintrihalide adduct and the lithium dialkylamide so as to form a tris(dialkylamido)alkyl tin product.

In some aspects, the techniques described herein relate to a method, wherein the method does not include forming an alkyltintrihalide-amine adduct.

In some aspects, the techniques described herein relate to a method, wherein the alkyltintrihalide-amine adduct is an alkyltintrihalide-(HNMe₂) adduct.

In some aspects, the techniques described herein relate to a method, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

In some aspects, the techniques described herein relate to a method, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

In some aspects, the techniques described herein relate to a method, wherein R is a vinyl, an allyl, a propynyl, a propenyl, or any isomer thereof.

In some aspects, the techniques described herein relate to a method, wherein the solvent includes at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, hexane, methanol, methyl t-butyl ether, methylene chloride, N-methyl-2-pyrrolidinone, pentane, petroleum ether, propanol, pyridine, tetrahydrofuran, toluene, triethylamine, water, xylene, any isomer thereof, or any combination thereof.

In some aspects, the techniques described herein relate to a method, wherein the lithium dialkylamide is a compound of the formula: LiN(R¹)₂, wherein: R¹ includes a C₁-C₃ alkyl.

In some aspects, the techniques described herein relate to a method, wherein the tris(dialkylamido)alkyl tin product is a compound of the formula:

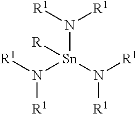

wherein R is a substituted C₁-C₅ alkyl, an unsubstituted C₁-C₅ alkyl, a substituted C₁-C₅ alkenyl, or an unsubstituted C₁-C₅ alkenyl; and wherein each R₁ is independently a C₁-C₃ alkyl.

In some aspects, the techniques described herein relate to a method including: obtaining an alkyltintrihalide; obtaining a solvent, a solution, or any combination thereof; and contacting the alkyltintrihalide with the solvent, the solution, or any combination thereof so as to form an alkyltintrihalide adduct; obtaining a lithium dialkylamide; and contacting the alkyltintrihalide adduct and the lithium dialkylamide so as to form a tris(dialkylamido)alkyl tin product.

In some aspects, the techniques described herein relate to a method, wherein the method does not include forming an alkyltintrihalide-amine adduct.

In some aspects, the techniques described herein relate to a method, wherein the alkyltintrihalide-amine adduct is an alkyltintrihalide-(HNMe₂) adduct.

In some aspects, the techniques described herein relate to a method, wherein the alkyltintrihalide is a compound of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

In some aspects, the techniques described herein relate to a method, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

In some aspects, the techniques described herein relate to a method, wherein R is a vinyl, an allyl, a propynyl, a propenyl, or any isomer thereof.

In some aspects, the techniques described herein relate to a method, wherein the solvent includes at least one of tetrahydrofuran (THF), dimethoxyethane (DME), or any combination thereof.

In some aspects, the techniques described herein relate to a method, wherein the solution includes at least one of hexane, pentane, toluene, or any combination thereof.

In some aspects, the techniques described herein relate to a method, wherein the solvent includes at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

In some aspects, the techniques described herein relate to a method, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

In some aspects, the techniques described herein relate to a method, wherein the lithium dialkylamide is a compound of the formula: LiN(R¹)₂, wherein: R¹ includes a C₁-C₃ alkyl.

In some aspects, the techniques described herein relate to a method, wherein the tris(dialkylamido)alkyl tin product is a compound of the formula:

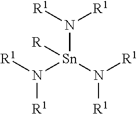

wherein R is a substituted C₁-C₅ alkyl, an unsubstituted C₁-C₅ alkyl, a substituted C₁-C₅ alkenyl, or an unsubstituted C₁-C₅ alkenyl; wherein each R¹ is independently a C₁-C₃ alkyl.

In some aspects, the techniques described herein relate to a composition including: an alkyltintrihalide adduct of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

In some aspects, the techniques described herein relate to a composition, wherein the solvent includes at least one of tetrahydrofuran (THF), dimethoxyethane (DME), hexane, or any combination thereof.

In some aspects, the techniques described herein relate to a composition, wherein the solvent includes at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, hexane, methanol, methyl t-butyl ether, methylene chloride, N-methyl-2-pyrrolidinone, pentane, petroleum ether, propanol, pyridine, tetrahydrofuran, toluene, triethylamine, water, xylene, any isomer thereof, or any combination thereof.

In some aspects, the techniques described herein relate to a composition including: a tris(dialkylamido)alkyl tin product of the formula:

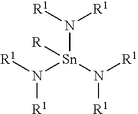

wherein R is a substituted C₁-C₅ alkyl, a substituted C₁-C₅ alkenyl, or an unsubstituted C₁-C₅ alkenyl; wherein each R1 is independently a C₁-C₃ alkyl.

In some aspects, the techniques described herein relate to a composition, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

In some aspects, the techniques described herein relate to a composition, wherein R is a vinyl, an allyl, a propynyl, a propenyl, or any isomer thereof.

In some aspects, the techniques described herein relate to a composition including: a reaction product of an alkyltintrihalide adduct and a lithium dialkylamide, wherein the reaction product including a compound of the formula:

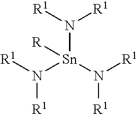

wherein R is a substituted C₁-C₅ alkyl, an unsubstituted C₁-C₅ alkyl, a substituted C₁-C₅ alkenyl, or an unsubstituted C₁-C₅ alkenyl; wherein each R¹ is independently a C₁-C₅ alkyl.

In some aspects, the techniques described herein relate to a composition, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

In some aspects, the techniques described herein relate to a composition, wherein R is a vinyl, an allyl, a propynyl, a propenyl, or any isomer thereof.

In some aspects, the techniques described herein relate to a composition including an atomic layer deposition precursor comprising an alkyltintrihalide of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

In some aspects, the techniques described herein relate to a composition, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

In some aspects, the techniques described herein relate to a composition, wherein R is a vinyl, an allyl, a propynyl, a propenyl, or any isomer thereof.

In some aspects, the techniques described herein relate to a composition including a chemical vapor deposition precursor comprising an alkyltintrihalide of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

In some aspects, the techniques described herein relate to a composition, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

In some aspects, the techniques described herein relate to a composition, wherein R is a vinyl, an allyl, a propynyl, a propenyl, or any isomer thereof.

In some aspects, the techniques described herein relate to a composition including, a compound of the formula:

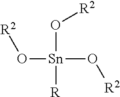

wherein:

R is a substituted C₁-C₅ alkyl, an unsubstituted C₁-C₅ alkyl, a substituted C₁-C₅ alkenyl, or an unsubstituted C₁-C₅ alkenyl;

R² is independently a substituted C₁-C₄ alkyl or an unsubstituted C₁-C₄ alkyl, wherein the substituted C₁-C₄ alkyl comprises a fluorine-containing substituent.

In some aspects, the techniques described herein relate to a composition, wherein the C₁-C₄ alkyl of R² is methyl, ethyl, n-propyl, iso-propyl, n-butyl, sec-butyl, or tert-butyl.

In some aspects, the techniques described herein relate to a composition, wherein the fluorine-containing substituent comprises —CH₂CF₃, —CH(CF₃)₂, or —(CHₐ)ₙ(CH_{b}F_{c})ₘ, wherein: a=0 to 3; b=0 to 2; c=1 to 3; n=0 to 3; m=1 to 4.

In some aspects, the techniques described herein relate to a composition, wherein —(CHₐ)ₙ(CH_{b}F_{c})ₘ is —CH₂F, —CH₂CH₂F, —CF₃, or —CF₂CF₃.

In some aspects, the techniques described herein relate to a composition, wherein R² is a saturated alkyl or an unsaturated alkyl.

In some aspects, the techniques described herein relate to a composition, wherein OR² is —OCH₂C≡CH or —OCH═CH₂.

In some aspects, the techniques described herein relate to a composition including a compound of the formula: RSn(OR²)₃, wherein: R is a substituted C₁-C₅ alkyl, an unsubstituted C₁-C₅ alkyl, a substituted C₁-C₅ alkenyl, or an unsubstituted C₁-C₅ alkenyl; R² is independently a substituted C₁-C₄ alkyl or an unsubstituted C₁-C₄ alkyl, wherein the substituted C₁-C₄ alkyl comprises a fluorine-containing substituent.

In some aspects, the techniques described herein relate to a composition, wherein the C₁-C₄ alkyl of R² is methyl, ethyl, n-propyl, iso-propyl, n-butyl, sec-butyl, or tert-butyl.

In some aspects, the techniques described herein relate to a composition, wherein the fluorine-containing substituent comprises —CH₂CF₃, —CH(CF₃)₂, or —(CHₐ)ₙ(CH_{b}F_{c})ₘ, wherein: a=0 to 3; b=0 to 2; c=1 to 3; n=0 to 3; m=1 to 4.

In some aspects, the techniques described herein relate to a composition, wherein —(CHₐ)ₙ(CH_{b}F_{c})ₘ is —CH₂F, —CH₂CH₂F, —CF₃, or —CF₂CF₃.

In some aspects, the techniques described herein relate to a composition, wherein R² is a saturated alkyl or an unsaturated alkyl.

In some aspects, the techniques described herein relate to a composition, wherein OR² is —OCH₂C≡CH or —OCH═CH₂.

### DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 depicts a non-limiting embodiment of a method of the present disclosure described herein.

FIG. 2 displays a ¹H-NMR of EtSnCl₃(THF)₂ recorded in CDCl₃.

FIG. 3 displays a solid-state 3-dimensional structure of EtSnCl₃(DME) as determined by X-ray crystallographic analysis.

FIG. 4 displays a ¹H-NMR of EtSnCl₃(DME) recorded in CDCl₃.

FIG. 5 displays ¹¹⁹Sn-NMR of EtSn(NMe₂)₃ synthesized from EtSnCl₃(THF)₂ recorded in C₆D₆.

FIG. 6 displays ¹¹⁹Sn-NMR of EtSn(NMe₂)₃ synthesized from EtSnCl₃(DME) recorded in C₆D₆.

FIG. 7 displays a solid-state 3-dimensional structure of vinylSnCl₃(DME) as determined by X-ray crystallographic analysis.

FIG. 8 displays ¹¹⁹Sn-NMR of vinylSnCl₃(DME) recorded in DME.

FIG. 9 displays a solid-state 3-dimensional structure of isopropenylSnCl₃(DME) as determined by X-ray crystallographic analysis.

FIG. 10 displays ¹H-NMR of isopropenylSnCl₃(DME) recorded in C₆D₆.

FIG. 11 displays ¹¹⁹Sn-NMR of isopropenylSn(NMe₂)₃ synthesized from isopropenylSnCl₃(DME) in a THF/hexanes solvent recorded in C₆D₆.

FIG. 12 displays ¹¹⁹Sn-NMR of isopropenylSn(NMe₂)₃ synthesized from isopropenylSnCl₃(DME) in a DME/hexanes solvent recorded in C₆D₆.

FIG. 13 displays ¹¹⁹Sn-NMR of isopropenylSn(NMe₂)₃ synthesized from isopropenylSnCl₃ in a hexanes solvent recorded in C₆D₆.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

All prior patents and publications referenced herein are incorporated by reference in their entireties.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

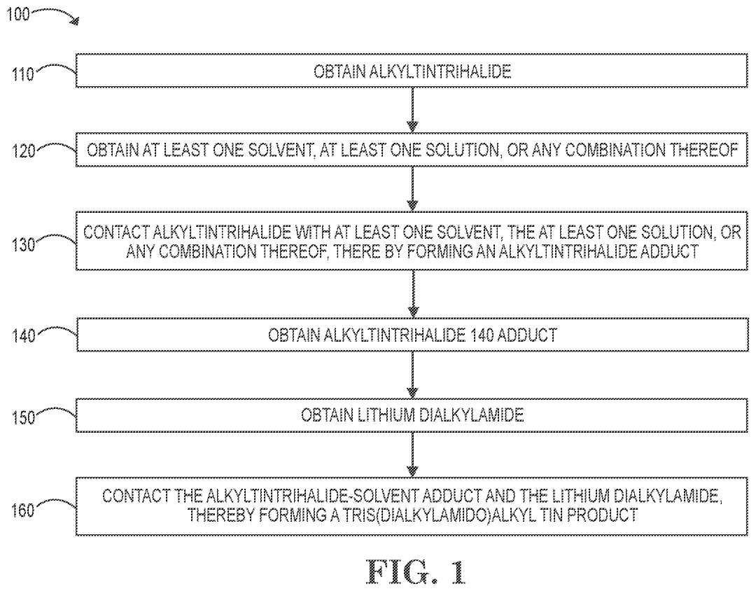

FIG. 1 depicts a non-limiting embodiment of a method 100 of the present disclosure described herein. The method 100 includes one or more of the following steps. A first step 110 includes obtaining an alkyltintrihalide. A second step 120 includes obtaining a solvent, a solution, or any combination thereof. A third step 130 includes contacting the alkyltintrihalide with the solvent, the solution, or any combination thereof, thereby forming an alkyltintrihalide adduct. A fourth step 140 includes obtaining an alkyltintrihalide adduct. A fifth step 150 includes obtaining a lithium dialkylamide. A sixth step 160 includes contacting the alkyltintrihalide adduct and the lithium dialkylamide, thereby forming a tris(dialkylamido)alkyl tin product.

The method 100 can include any combination of the steps. For example, in some embodiments, the method 100 can be the first step 110, second step 120, and the third step 130. In some embodiments, the method 100 can be the fourth step 140, the fifth step 150, and the sixth step 160. In some embodiments, the method 100 can be the first step 110, second step 120, and the third step 130, the fourth step 140, the fifth step 150, and the sixth step 160.

In some embodiments, the method 100 does not include forming an alkyltintrihalide-amine adduct. In some embodiments, the alkyltintrihalide-amine adduct is an alkyltintrihalide-(HNMe₂) adduct. For example, in some embodiments, the alkyltintrihalide-(HNMe₂) adduct has two amines —(HNMe₂)₂.

The present disclosure is directed to adducts of alkyltintrihalides, including solvent-based adducts of alkyltintrihalides, for production of high-purity (e.g., greater than 95% purity) extreme ultraviolet atomic layer deposition precursors. In some embodiments, the purity is greater than 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, or 99.99%.

In some embodiments, the alkyltintrihalide (e.g., see the first step 110) is a compound of the formula RSnX₃. In some embodiments, R is a substituted C₁-C₅ alkyl, an unsubstituted C₁-C₅ alkyl, a substituted C₁-C₅ alkenyl, an unsubstituted C₁-C₅ alkenyl, a substituted C₁-C₅ alkynyl, or an unsubstituted C₁-C₅ alkynyl. In some embodiments, X is Cl, Br, or I.

In some embodiments, the solvent (e.g., see the second step 120) includes at least one of tetrahydrofuran (THF), dimethoxyethane (DME), or any combination thereof.

In some embodiments, the solution (e.g., see the second step 120) includes at least one of hexane, pentane, toluene, or any combination thereof.

In some embodiments, the solvent includes at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

In some embodiments, the alkyltintrihalide adduct (e.g., see the third step 130 and/or the fourth step 140) is a compound of the formula RSnX₃·(solv)ₙ. In some embodiments, R is a substituted C₁-C₅ alkyl, an unsubstituted C₁-C₅ alkyl, a substituted C₁-C₅ alkenyl, or an unsubstituted C₁-C₅ alkenyl. X is Cl, Br, or I. In some embodiments, solv is a solvent. In some embodiments, n is at least 1.

In some embodiments, the lithium dialkylamide (e.g., see the fifth step 150) is a compound of the formula LiN(R¹)₂. In some embodiments, R¹ is a C₁-C₃ alkyl.

In some embodiments, the present disclosure includes a composition including the tris(dialkylamido)alkyl tin product (e.g., see the sixth step 160).

In some embodiments, the present disclosure includes a composition including a reaction product of an alkyltintrihalide adduct and a lithium dialkylamide.

In some embodiments, the tris(dialkylamido)alkyl tin product (e.g., see the sixth step 160) is a compound of the formula:

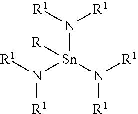

In some embodiments, R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, CFH₂, or an alkoxy.

In some embodiments, R is a vinyl, an allyl, a propynyl, a propenyl, or any isomer thereof.

In some embodiments, R is a substituted C₁-C₅ alkyl, a substituted C₁-C₅ alkenyl, or an unsubstituted C₁-C₅ alkenyl. In some embodiments, each R¹ is independently a C₁-C₃ alkyl.

In some embodiments, each R and each R¹ can be independently chosen from straight or branched chain alkyl groups including methyl, ethyl, propyl, isopropyl, n-butyl, t-butyl, sec-butyl, n-pentyl, isopentyl, or sec-pentyl groups. In one specific embodiment, each R and each R¹ is independently chosen from C₁-C₃ alkyl group such as a methyl, ethyl, or propyl group. In some embodiments, R or R¹ is chosen from C₁-C₅ alkyl groups, which can be substituted or unsubstituted straight or branched chain alkyl group. For example, R or R¹ may be a straight or branched chain alkyl group including methyl, ethyl, propyl, isopropyl, n-butyl, t-butyl, sec-butyl, n-pentyl, isopentyl, or sec-pentyl groups. In addition, R or R¹ can be a cyclic C₁-C₅ group such as a cyclopropyl group. Also, R or R¹ may be an unsaturated C₁-C₅ group such as a vinyl group or an acetylenyl group. Any of the R or R¹ groups may be further substituted, such as with one or more halogen groups or ether groups. For example, R or R¹ may be a fluorinated alkyl group having the formula —(CHₐ)ₙ(CH_{b}F_{c})ₘ, wherein a=0 to 3; b=0 to 2; c=1 to 3; n=0 to 3; m=1 to 4, including a monofluorinated C₁-C₅ alkyl group, such as a —CH₂F or —CH₂CH₂F group, and a perfluorinated C₁-C₅ group, such as a —CF₃ or —CF₂CF₃ group. Alternatively, R or R¹ may be an alkylether group, wherein the alkyl portion is a C₁-C₅ alkyl group. In one specific embodiment, each R and each R¹ is methyl, ethyl, or isopropyl.

In some embodiments, R or R¹ may be an alkyl, alkenyl, alkynyl, alkoxide, carboxylate, ether, nitrile, or an imide.

In some embodiments, R or R¹ may be a C₁-C₅ alkyl (methyl, ethyl, n-propyl, isopropyl, cyclopropyl, sec-butyl, n-butyl, tert-butyl, iso-amyl, cyclopentadienyl, vinyl, ethynyl, propynyl, isopropenyl, or acetyl).

In some embodiments, R or R¹ may be a C₆-Cₙ phenyl, including substituted phenyls or substituted cyclopentadienylides (e.g., indene).

In some embodiments, R or R¹ may be a functionalized alkyl, including —CF₃, —CF₂H, —CFH₂, CF₃CH₂, CF₂HCH₂, CFH₂CH₂, ICH₂CH₂ (iodoethane), CH₃₀CH₂, CH₃CH₂OCH₂—, CH₃CH₂OCH₂CH₂—, CH₃₀CH₂CH₂—, or —C≡N.

In some embodiments, R or R¹ may be carboxylates, including CF₃CO2 (trifluoroacetate) or CH₃CO2 (acetate).

In some embodiments, R or R¹ may be an alkoxide (CXₙH₃₋ₙCXₘH₂₋ₘO—, where X═F, Cl, Br, I and n,m=0-3). For example, R or R¹ may be CF₃CH₂O— (trifluoroethoxide), CH₃O— (methoxide), CH₃CH₂O— (ethoxide), (CH₃)₂CHO— (isopropoxide), (CH₃)₃CO— (tert-butoxide), or HC≡CO— (propargyl alkoxide).

In some embodiments, R or R¹ may be any combination of the compounds described in the present disclosure (e.g., fluoroethers or fluoroalkoxides).

Some embodiments relate to a composition. In some embodiments, the composition comprises an atomic layer deposition precursor comprising an alkyltintrihalide of the formula: RSnX₃.

In some embodiments, R is a substituted C₁-C₅ alkyl, an unsubstituted C₁-C₅ alkyl, a substituted C₁-C₅ alkenyl, or an unsubstituted C₁-C₅ alkenyl. In some embodiments, X is Cl, Br, or I.

In some embodiments, R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

In some embodiments, R is a vinyl, an allyl, a propynyl, a propenyl, or any isomer thereof.

Some embodiments relate to a composition. In some embodiments, the composition comprises a chemical vapor deposition precursor comprising an alkyltintrihalide of the formula: RSnX₃.

In some embodiments, R is a substituted C₁-C₅ alkyl, an unsubstituted C₁-C₅ alkyl, a substituted C₁-C₅ alkenyl, or an unsubstituted C₁-C₅ alkenyl. In some embodiments, X is Cl, Br, or I.

In some embodiments, R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

In some embodiments, R is a vinyl, an allyl, a propynyl, a propenyl, or any isomer thereof.

Some embodiments relate to a composition comprising a compound of the formula:

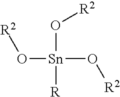

In some embodiments, R is a substituted C₁-C₅ alkyl, an unsubstituted C₁-C₅ alkyl, a substituted C₁-C₅ alkenyl, or an unsubstituted C₁-C₅ alkenyl.

In some embodiments, R² is independently a substituted C₁-C₄ alkyl or an unsubstituted C₁-C₄ alkyl. In some embodiments, the substituted C₁-C₄ alkyl comprises a fluorine-containing substituent.

In some embodiments, the C₁-C₄ alkyl of R² is methyl, ethyl, n-propyl, iso-propyl, n-butyl, sec-butyl, or tert-butyl.

In some embodiments, the fluorine-containing substituent comprises —CH₂CF₃, —CH(CF₃)₂, or —(CHₐ)ₙ(CH_{b}F_{c})ₘ, wherein: a=0 to 3; b=0 to 2; c=1 to 3; n=0 to 3; m=1 to 4.

In some embodiments, —(CHₐ)ₙ(CH_{b}F_{c})ₘ is —CH₂F, —CH₂CH₂F, —CF₃, or —CF₂CF₃.

In some embodiments, R² is a saturated alkyl or an unsaturated alkyl.

In some embodiments, OR² is —OCH₂C≡CH or —OCH═CH₂.

Some embodiments relate to a composition comprising a compound of the formula: RSn(OR²)₃.

In some embodiments, R is a substituted C₁-C₅ alkyl, an unsubstituted C₁-C₅ alkyl, a substituted C₁-C₅ alkenyl, or an unsubstituted C₁-C₅ alkenyl.

In some embodiments, R² is independently a substituted C₁-C₄ alkyl or an unsubstituted C₁-C₄ alkyl. In some embodiments, the substituted C₁-C₄ alkyl comprises a fluorine-containing substituent.

In some embodiments, the C₁-C₄ alkyl of R² is methyl, ethyl, n-propyl, iso-propyl, n-butyl, sec-butyl, or tert-butyl.

In some embodiments, the fluorine-containing substituent comprises —CH₂CF₃, —CH(CF₃)₂, or —(CHₐ)ₙ(CH_{b}F_{c})ₘ, wherein: a=0 to 3; b=0 to 2; c=1 to 3; n=0 to 3; m=1 to 4.

In some embodiments, —(CHₐ)ₙ(CH_{b}F_{c})ₘ is —CH₂F, —CH₂CH₂F, —CF₃, or —CF₂CF₃.

In some embodiments, R² is a saturated alkyl or an unsaturated alkyl.

In some embodiments, OR² is —OCH₂C≡CH or —OCH═CH₂.

### EXAMPLES

### Example 1: Synthesis of EtSnCl₃(THF)₂

In a nitrogen-filled glovebox, EtSnCl₃ (0.500 g, 1.96 mmol) was placed in a 40 mL vial and diluted with hexanes (2 mL). Tetrahydrofuran (0.71 g, 9.84 mmol) was added dropwise to the EtSnCl₃ solution, resulting in the immediate production of a white precipitate. Upon complete addition of THF, the vial was placed in the freezer at −35° C. for 1 hour, the mother liquor pipetted away, and the remaining white solid warmed to room temperature and dried under reduced pressure. Upon warming, the product melted to form a colorless liquid. Mass: 3.12 g, 99.7% yield. ¹H-NMR (400 MHz, CDCl₃, 298K): 1.32 (t, 3H); 1.74 (t, 8H); 2.13 (q, 2H); 3.67 (t, 8H) ppm; ¹³C{¹H}-NMR (100 MHz, CDCl₃, 298K): 9.46; 25.07; 29.20; 68.32 ppm; ¹¹⁹Sn{¹H}-NMR (149 MHz, CDCl₃, 298K): −150.98 ppm.

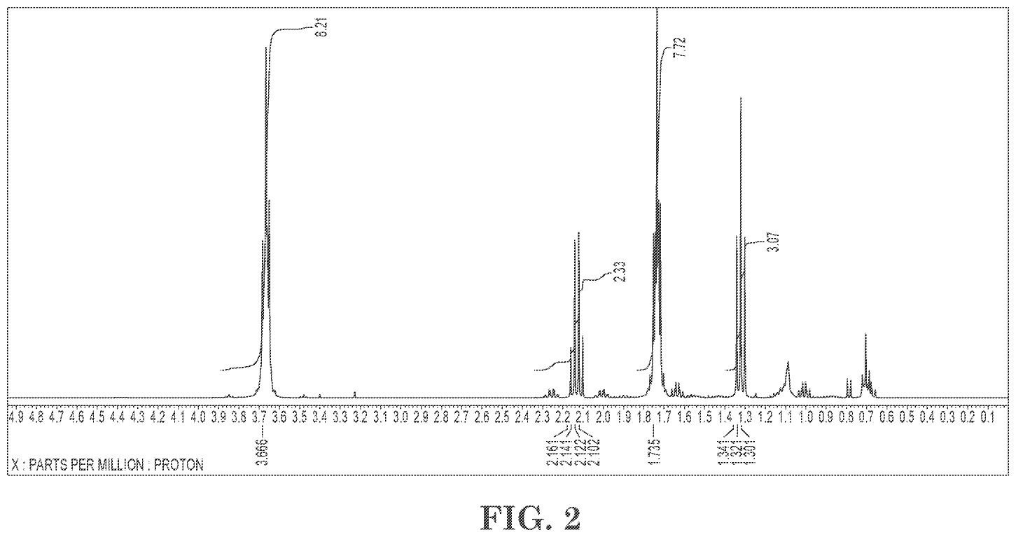

FIG. 2 displays a ¹H-NMR of EtSnCl₃(THF)₂ recorded in CDCl₃.

### Example 2: Synthesis of EtSnCl₃(DME)

In a nitrogen-filled glovebox, EtSnCl₃ (0.500 g, 1.96 mmol) was placed in a 40 mL vial and diluted with hexanes (2 mL). Dimethoxyethane (1.13 g, 12.5 mmol) was added dropwise to the EtSnCl₃ solution, resulting in the immediate production of a white precipitate. Upon complete addition of DME, the vial was placed in the freezer at −35° C. for 1 hour, the mother liquor pipetted away, and the remaining white solid warmed to room temperature and dried under reduced pressure. Mass: 2.70 g, 98.8% yield). M.P.: 61.9° C. (by DSC). X-ray quality crystals were grown from cooling a saturated DME solution of EtSnCl₃(DME) at −35° C. ¹H-NMR (400 MHz, CDCl₃, 298K): 1.46 (t, 3H); 2.28 (q, 2H); 3.39 (s, 6H); 3.56 (s, 4H) ppm; ¹³C{¹H}-NMR (100 MHz, CDCl₃, 298K): 9.54; 28.22; 59.26; 71.44 ppm; ¹¹⁹Sn{¹H}-NMR (149 MHz, CDCl₃, 298K): −49.63 ppm.

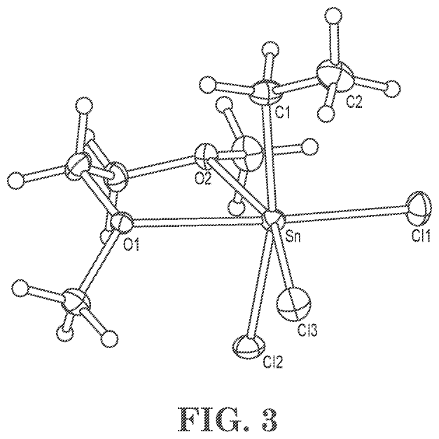

FIG. 3 displays a solid-state 3-dimensional structure of EtSnCl₃(DME) as determined by X-ray crystallographic analysis. Table 1 displays the crystal data and structural refinement for EtSnCl₃(DME).

TABLE 1
Crystal data and structure refinement for EtSnCl3(DME).

| Empirical formula | C6 H15 Cl3 O2 Sn | |
|---|---|---|
| Molecular formula | C6 H15 Cl3 O2 Sn | |
| Formula weight | 344.22 | |
| Temperature | 100.00 | K |
| Wavelength | 0.71073 | Å |
| Crystal system | Monoclinic | |
| Space group | P 1 21/n 1 | |
| Unit cell dimensions | a = 8.4452(4) Å; α = 90° | |
| | b = 10.5566(4) Å; β = 103.3670 (10)° | |
| | c = 13.7754(6) Å; γ = 90° | |
| Volume | 1194.84(9) | Å³ |
| Z | 4 | |
| Density (calculated) | 3 | |
| | 1.914 | Mg/m³ |
| Absorption coefficient | 2.774 | mm⁻¹ |
| F(000) | 672 | |
| Crystal size | 0.175 × 0.15 × 0.13 | mm³ |

| | |
|---|---|
| Crystal color, habit | colorless block |
| Theta range for data collection | 2.456 to 26.752° |
| Index ranges | −10 <= h <= 10, −13 <= |
| | k <= 13, −17 <= l <= 17 |
| Reflections collected | 20456 |
| Independent reflections | 2539 [R(int) = 0.0358] |
| Completeness to theta = 25.242° | 100.0% |
| Absorption correction | Semi-empirical from equivalents |
| Max. and min. transmission | 0.4910 and 0.4096 |
| Refinement method | Full-matrix least-squares on F² |
| Data/restraints/parameters | 2539/0/113 |
| Goodness-of-fit on F² | 1.076 |
| Final R indices [I > 2sigma(I)] | R1 = 0.0131, wR2 = 0.0303 |
| R indices (all data) | R1 = 0.0146, wR2 = 0.0307 |
| Extinction coefficient | 0.0025(2) |
| Largest diff. peak and hole | 0 0.336 d −0.266 e · Å⁻³ |

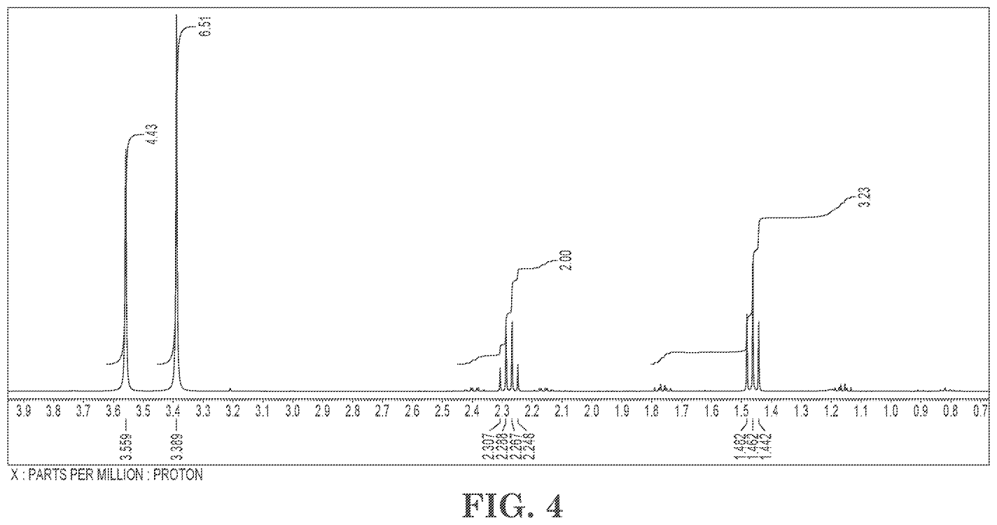

FIG. 4 displays a ¹H-NMR of EtSnCl₃(DME) recorded in CDCl₃.

### Example 3: Synthesis of EtSn(NMe₂)₃ Using EtSnCl₃(THF)₂

In a nitrogen-filled glovebox, EtSnCl₃ (3.0 g, 11.8 mmol) was placed in a 40 mL vial and diluted with tetrahydrofuran (4 mL, 49.2 mmol), resulting in an exotherm and presentation as a colorless solution. Separately, LiNMe₂ (1.89 g, 37.1 mmol) was placed in a 40 mL amber vial equipped with a magnetic stir bar and diluted with hexanes (10 mL). Upon cooling to room temperature, the EtSnCl₃(THF)₂ solution was added dropwise to the LiNMe₂ mixture with stirring over the course of 5 minutes, resulting in an exotherm and the production of a white precipitate, at which point the resulting white mixture was stirred overnight.

The following morning the reaction presented as a white mixture, was filtered through a syringe filter, and the organic solution dried under reduced pressure to yield a pale-yellow solid and white solid. ¹H-, ¹³C-, and ¹¹⁹Sn-NMR recorded on a concentrated C₆D₆ solution of the product confirm the target molecule has been synthesized in >98.5% initial purity. A long-duration ¹¹⁹Sn-NMR experiment collecting over 10 thousand scans was performed to confirm Et₂Sn(NMe₂)₂ (27.7 ppm) and Et₃Sn(NMe₂) (52.6 ppm) were non-detect by ¹¹⁹Sn-NMR.

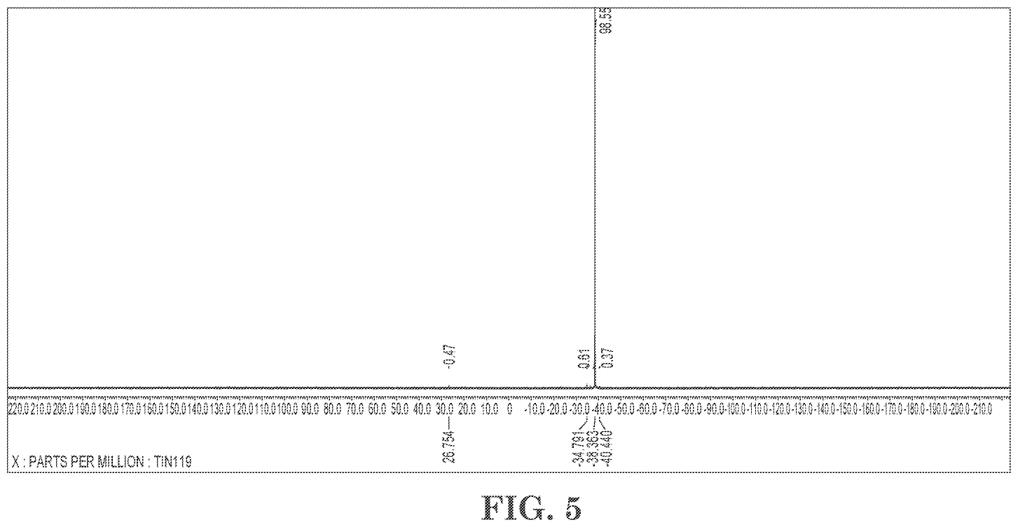

FIG. 5 displays ¹¹⁹Sn-NMR of EtSn(NMe₂)₃ synthesized from EtSnCl₃(THF)₂ recorded in C₆D₆.

### Example 4: Synthesis of EtSn(NMe₂)₃ Using EtSnCl₃(DME)

In a nitrogen-filled glovebox, EtSnCl₃ (3.0 g, 11.8 mmol) was added to dimethoxyethane (5 mL, 59.0 mmol), resulting in an exotherm and presentation as a colorless solution. Separately, LiNMe₂ (1.89 g, 37.1 mmol) was placed in a 40 mL amber vial equipped with a magnetic stir bar and diluted with a 1:1 solution of DME/hexanes (10 mL). Upon cooling to room temperature, the EtSnCl₃(DME) solution was added dropwise to the LiNMe₂ mixture with stirring over the course of 5 minutes, resulting in an exotherm and the production of a white precipitate, at which point the resulting white mixture was stirred overnight.

The following morning the reaction presented as a white mixture and the solvent was removed under reduced pressure to produce a white matrix. The product was extracted with hexanes (10 mL), the resulting white mixture filtered through a 0.2 μm syringe filter, and the organic solution dried under reduced pressure to yield the product as a pale-yellow liquid and isolated to yield a product (1.44 g, 5.14 mmol) for a 43.6% yield. ¹H-, ¹³C-, and ¹¹⁹Sn-NMR were recorded on a concentrated C₆D₆ solution of the product to confirm the target molecule has been synthesized in >95% initial purity.

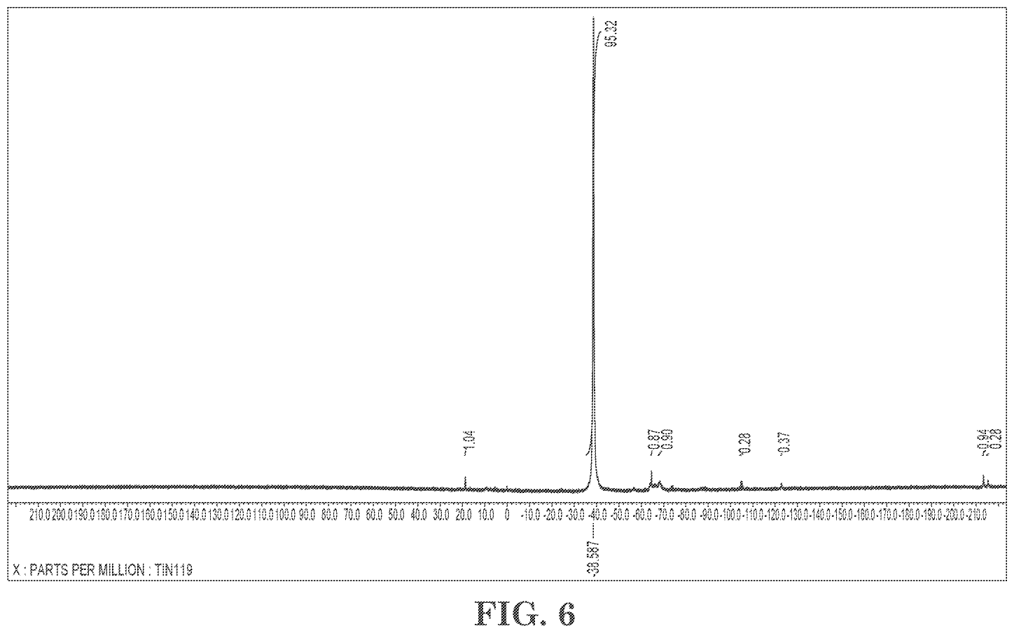

FIG. 6 displays ¹¹⁹Sn-NMR of EtSn(NMe₂)₃ synthesized from EtSnCl₃(DME) recorded in C₆D₆. A long-duration ¹¹⁹Sn-NMR experiment collecting over 10 thousand scans was performed to confirm Et₂Sn(NMe₂)₂ (27.7 ppm) and Et₃Sn(NMe₂) (52.6 ppm) were non-detected by ¹¹⁹Sn-NMR.

### Example 5: Synthesis of VinylSnCl₃(DME)

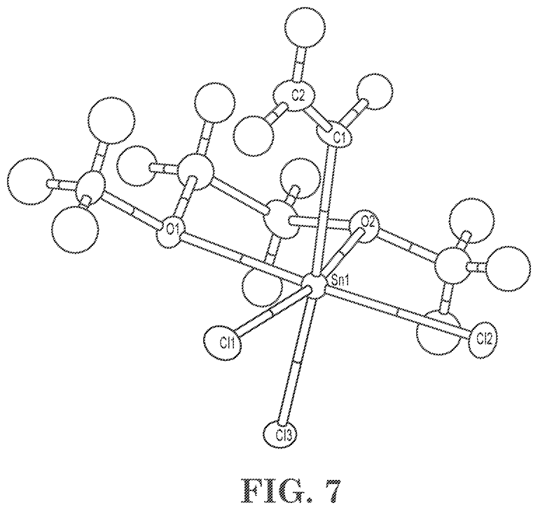

FIG. 7 displays a solid-state 3-dimensional structure of vinylSnCl₃(DME) as determined by X-ray crystallographic analysis. Table 2 displays the crystal data and structural refinement for vinylSnCl₃(DME).

TABLE 2
Crystal data and structure refinement for vinylSnCl3(DME).

| Empirical formula | C6 H13 Cl3 O2 Sn | |
|---|---|---|
| Molecular formula | C6 H13 Cl3 O2 Sn | |
| Formula weight | 342.20 | |
| Temperature | 100.00 | K |
| Wavelength | 0.71073 | Å |
| Crystal system | Monoclinic | |
| Space group | P 1 21/c 1 | |
| Unit cell dimensions | a = 15.1719(4); α = 90° | |
| | b = 12.6777(3) Å; β = 100.5480 (10)° | |
| | c = 12.4933(3) Å; γ = 90° | |
| Volume | 2362.41(10) | Å³ |
| Z | 8 | |
| Density (calculated) | 1.914 | Mg/m³ |
| Absorption coefficient | 2.806 | mm⁻¹ |
| F(000) | 1328 | |
| Crystal size | 0.15 × 0.08 × 0.05 | mm³ |

| | |
|---|---|
| Crystal color, habit | colorless block |
| Theta range for data collection | 2.523 to 26.366° |
| Index ranges | −18 <= h <= 18, −15 <= |
| | k <= 15, −15 <= l <= 15 |
| Reflections collected | 54445 |
| Independent reflections | 4828 [R(int) = 0.0484] |
| Completeness to theta = 25.242° | 99.9% |
| Absorption correction | Semi-empirical from equivalents |
| Max. and min. transmission | 0.2607 and 0.2225 |
| Refinement method | Full-matrix least-squares on F² |
| Data/restraints/parameters | 4828/0/222 |
| Goodness-of-fit on F² | 1.043 |
| Final R indices [I > 2sigma(I)] | R1 = 0.0172, wR2 = 0.0325 |
| R indices (all data) | R1 = 0.0233, wR2 = 0.0339 |
| Extinction coefficient | 0.00040(4) |
| Largest diff. peak and hole | 0.398 and −0.291 e · Å⁻³ |

In a nitrogen-filled glovebox, dimethoxyethane (DME) (2.6 g, 28.8 mmol) was placed in a 40 mL vial, and vinylSnCl₃ (2.0 g, 7.93 mmol) was added dropwise resulting in the immediate production of a white precipitate and exotherm. X-ray quality crystals were grown from cooling a saturated DME solution of vinylSnCl₃(DME) that was layered with hexanes at −35° C.

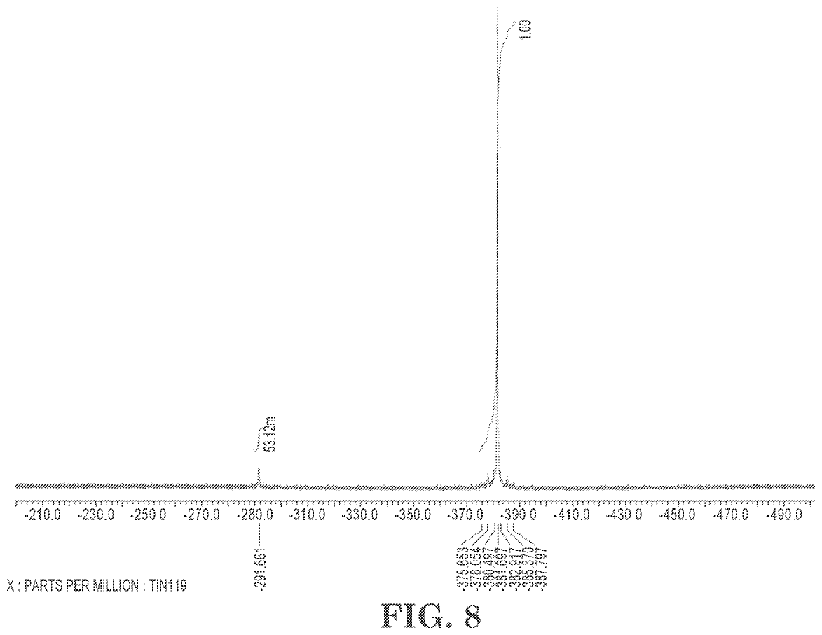

FIG. 8 displays ¹¹⁹Sn-NMR of vinylSnCl₃(DME) recorded in DME. ¹¹⁹Sn{¹H}-NMR (149 MHz, DME, 298K): −381.7 ppm (impurity from starting vinylSnCl₃ at −291.6 ppm).

### Example 6: Synthesis of VinylSn(NMe₂)₃ Using VinylSnCl₃(DME) in DME/Hexanes

In a nitrogen-filled glovebox, vinylSnCl₃ (15 g, 59.4 mmol) was added dropwise to dimethoxyethane (15 mL, 144 mmol) within a 250 mL flask fitted with a stir bar. The addition 50 mL of hexanes was added to the flask and caused the vinylSnCl₃ adduct to crash out of solution and the mixture to become a suspension. LiNMe₂ (9.38 g, 184 mmol) solid was added in small portions over 3.5 hours. The resulting white mixture was stirred overnight.

The volatiles were removed under vacuum. The residue was extracted with hexanes and filtered through a polypropylene (PP) fritted filter. The organic solution was dried under reduced pressure to yield the product as a pale-yellow liquid. ¹¹⁹Sn-NMR recorded on a concentrated C₆D₆ solution of the product showed a.

### Example 7: Synthesis of IsopropenylSnCl₃(DME)

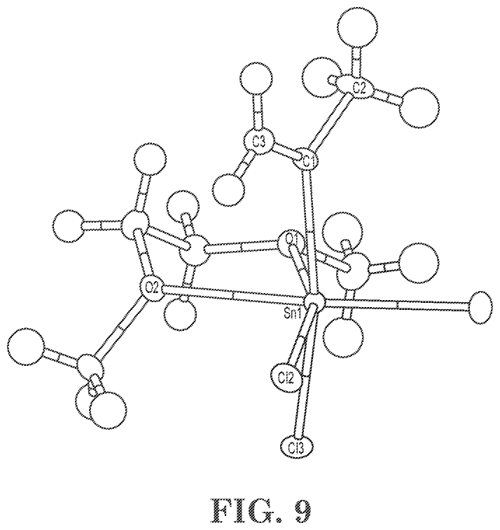

FIG. 9 displays a solid-state 3-dimensional structure of isopropenylSnCl₃(DME) as determined by X-ray crystallographic analysis. Table 3 displays the crystal data and structural refinement for isopropenylSnCl₃(DME).

TABLE 3
Crystal data and structure refinement for isopropenylSnCl3(DME).

| Empirical formula | C7 H15 Cl3 O2 Sn | |
|---|---|---|
| Molecular formula | C7 H15 Cl3 O2 Sn | |
| Formula weight | 356.23 | |
| Temperature | 100.00 | K |
| Wavelength | 0.71073 | Å |
| Crystal system | Triclinic | |
| Space group | P-1 | |
| Unit cell dimensions | a = 7.0720(3); α = 84.4810(10)° | |
| | b = 7.2438(3) Å; β = 83.0180(10)° | |
| | c = 12.8313(5) Å; γ = 71.6970(10)° | |
| Volume | 618.27(4) | Å³ |
| Z | 2 | |
| Density (calculated) | 1.914 | Mg/m³ |
| Absorption coefficient | 2.685 | mm⁻¹ |
| F(000) | 348 | |
| Crystal size | 0.18 × 0.15 × 0.06 | mm³ |

| | |
|---|---|
| Crystal color, habit | colorless block |
| Theta range for data collection | 2.968 to 26.367° |
| Index ranges | −8 <= h <= 8, −9 <= |
| | k <= 9, −16 <= l <= 15 |
| Reflections collected | 13723 |
| Independent reflections | 2517 [R(int) = 0.0314] |
| Completeness to theta = 25.242° | 99.7% |
| Absorption correction | Semi-empirical from equivalents |
| Max. and min. transmission | 0.2607 and 0.2097 |
| Refinement method | Full-matrix least-squares on F² |
| Data/restraints/parameters | 2517/0/122 |
| Goodness-of-fit on F² | 1.210 |
| Final R indices [I > 2sigma(I)] | R1 = 0.0164, wR2 = 0.0438 |
| R indices (all data) | R1 = 0.0167, wR2 = 0.0439 |
| Extinction coefficient | 0.0093(7) |
| Largest diff. peak and hole | 0.542 and −0.366 e · Å⁻³ |

In a nitrogen-filled glovebox, isopropenylSnCl₃ (2.0 g, 7.51 mmol) was placed in a 40 mL vial and diluted with hexanes (7 mL). Dimethoxyethane (1.73 g, 19.1 mmol) was added dropwise to the isopropenylSnCl₃ solution, resulting in the immediate production of a white precipitate. Upon complete addition of DME, the vial was stirred at room temperature for 1 hour. The volatiles were removed under vacuum, and the white solids washed with hexanes. The remaining white solid was then dried under reduced pressure. Mass: 2.63 g, (97.7% yield). X-ray quality crystals were grown from cooling a saturated DME solution of isopropenylSnCl₃(DME) that was layered with hexanes at −35° C.

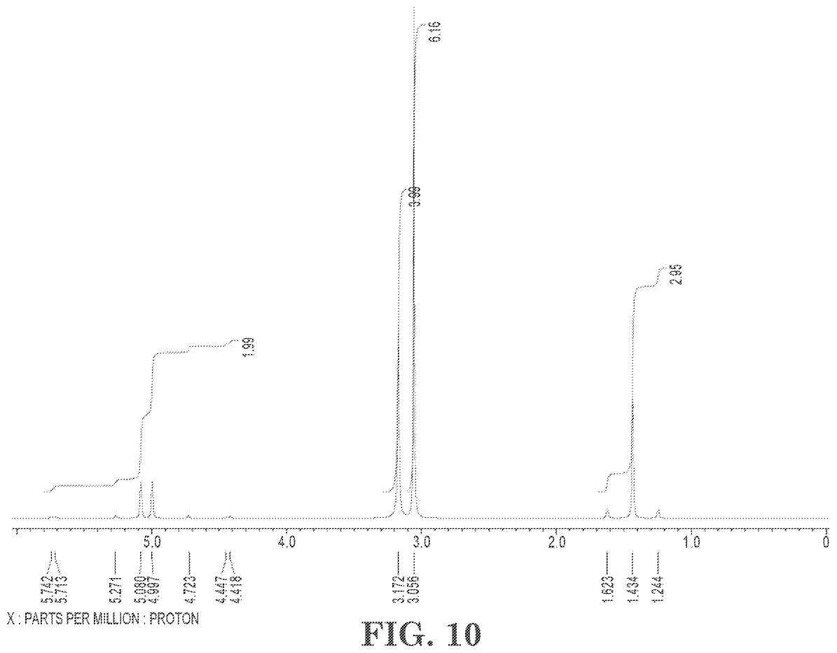

FIG. 10 displays ¹H-NMR of isopropenylSnCl₃(DME) recorded in C₆D₆. ¹H-NMR (400 MHz, C₆D₆, 298K): 1.43 (s, 3H); 3.06 (s, 6H); 3.17 (s, 4H); 4.99 and 5.08 (s, 2H) ppm.

For ¹³C-NMR of isopropenylSnCl₃(DME) recorded in C₆D₆. ¹³C{¹H}-NMR (100 MHz, C₆D₆, 298K): 22.72; 58.73; 71.35; 131.34; 146.96 ppm.

For ¹¹⁹Sn-NMR of isopropenylSnCl₃(DME) recorded in C₆D₆. ¹¹⁹Sn{¹H}-NMR (149 MHz, C₆D₆, 298K): −106.9 ppm.

### Example 8: Synthesis of IsopropenylSn(NMe₂)₃ Using IsopropenylSnCl₃(DME) in THF/Hexanes

In a nitrogen-filled glovebox, isopropenylSnCl₃ (2.0 g, 7.51 mmol) was added to dimethoxyethane (5 mL, 59.0 mmol), resulting in an exotherm and presentation as a white solid in solution. Excess DME was removed under vacuum, and the solid suspended in 10 mL of hexanes. Separately, LiNMe₂ (1.14 g, 22.5 mmol) was placed in a 40 mL amber vial equipped with a magnetic stir bar and diluted with a 1:1 solution of THF/hexanes (12 mL). The LiNMe₂ mixture was added dropwise to the isopropenylSnCl₃(DME) suspension with stirring, resulting in an exotherm and the production of a white precipitate, at which point the resulting white mixture was stirred for 1 hour.

The reaction presented as a pale-yellow cloudy mixture. The resulting mixture was filtered through a 0.2 μm syringe filter, and the organic solution dried under reduced pressure to yield the product as a pale-yellow liquid. Isolated 1.4 g, (4.79 mmol) for a 63.9% yield. ¹H-, ¹³C-, and ¹¹⁹Sn-NMR recorded on a concentrated C₆D₆ solution of the product confirm the target molecule has been synthesized in >85% initial purity.

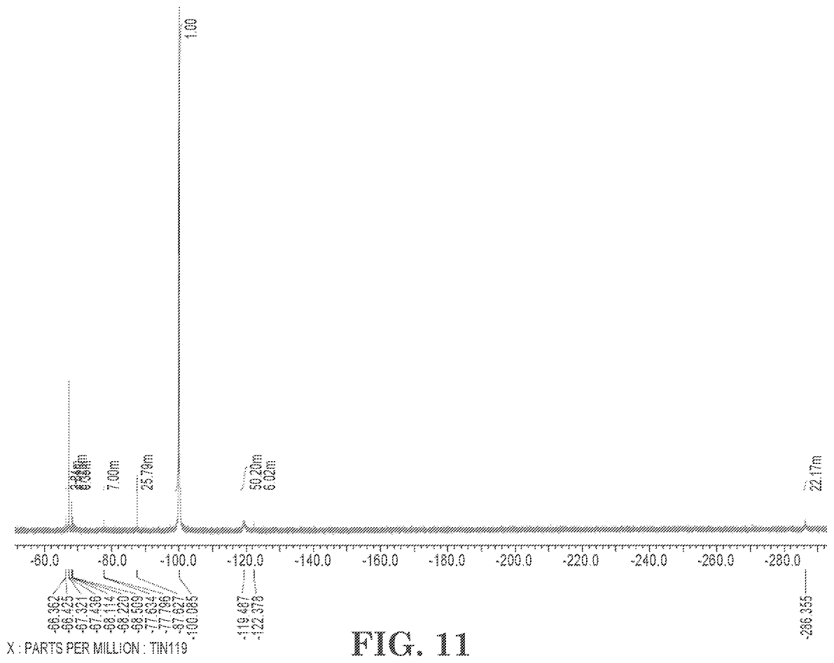

FIG. 11 displays ¹¹⁹Sn-NMR of isopropenylSn(NMe₂)₃ synthesized from isopropenylSnCl₃(DME) in a THF/hexanes solvent recorded in C₆D₆.

### Example 9: Synthesis of IsopropenylSn(NMe₂)₃ Using IsopropenylSnCl₃(DME) in DME/Hexanes

In a nitrogen-filled glovebox, isopropenylSnCl₃(DME) adduct (2.6 g, 7.25 mmol) was dissolved in dimethoxyethane (5 mL, 59.0 mmol) within a 40 mL amber vial fitted with a stir bar. Separately, LiNMe₂ (1.13 g, 22.2 mmol) was suspended in a 1:1 solution of DME/hexanes (10 mL). The LiNMe₂ mixture was added dropwise to the isopropenylSnCl₃(DME) suspension with stirring, resulting in an exotherm and the production of a white precipitate, at which point the resulting white mixture was stirred overnight.

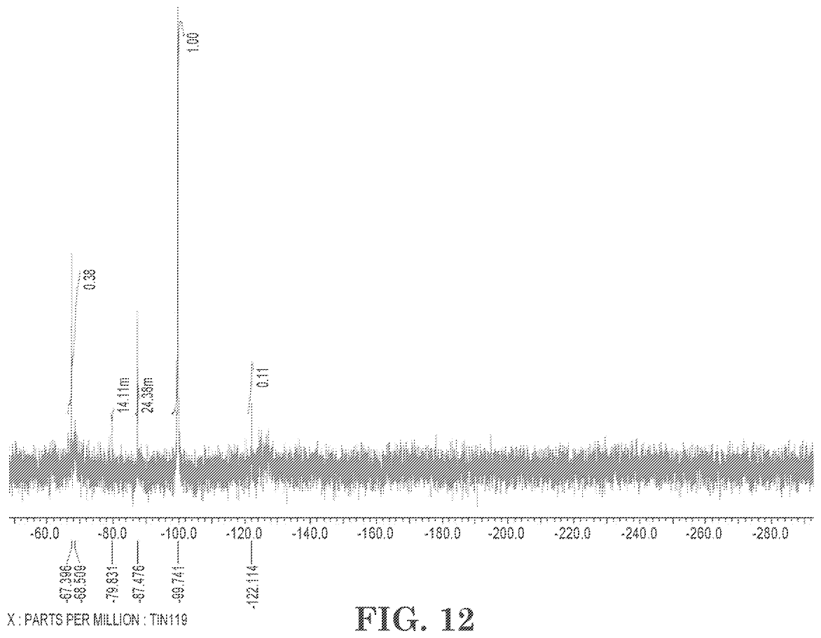

The volatiles were removed under vacuum. The residue was extracted with hexanes and filtered through a polypropylene (PP) fritted filter. The organic solution was dried under reduced pressure to yield the product as a pale-yellow liquid. Isolated 1.08 g, (3.69 mmol) for a 50.9% yield. ¹H-, ¹³C-, and ¹¹⁹Sn-NMR recorded on a concentrated C₆D₆ solution of the product confirm the target molecule has been synthesized in >65% initial purity. FIG. 12 displays ¹¹⁹Sn-NMR of isopropenylSn(NMe₂)₃ synthesized from isopropenylSnCl₃(DME) in a DME/hexanes solvent recorded in C₆D₆.

### Example 10: Synthesis of IsopropenylSn(NMe₂)₃ Using IsopropenylSnCl₃(DME) in Hexanes

In a nitrogen-filled glovebox, isopropenylSnCl₃ (2.6 g, 7.25 mmol) in hexanes (5 mL) was added to a 40 mL amber vial with stir bar where LiNMe₂ (1.15 g, 22.7 mmol) was suspended in hexanes (10 mL). An exotherm and the production of a white precipitate were observed, and the resulting mixture was stirred overnight.

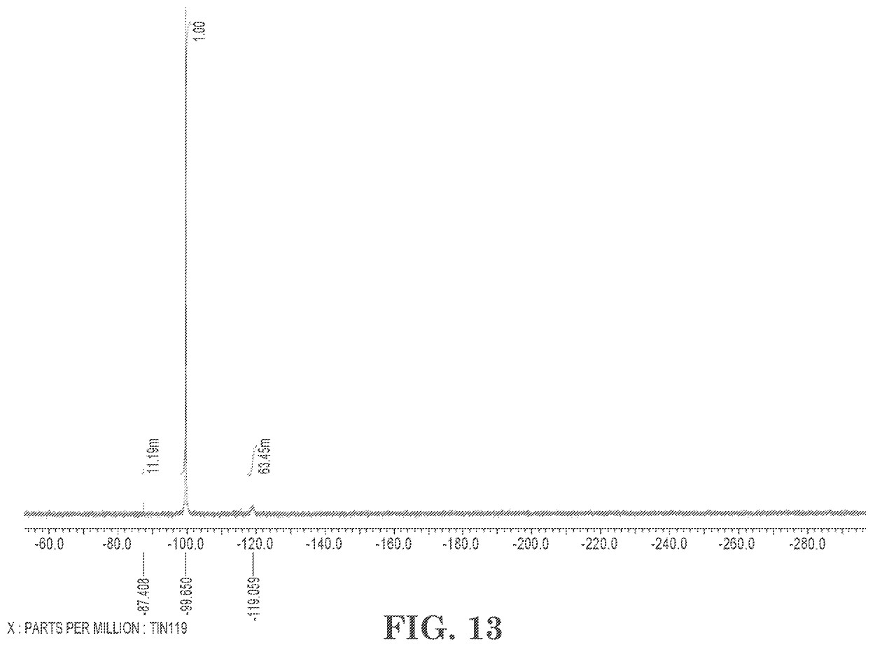

The mixture was filtered through a polypropylene (PP) fritted filter. Solids were extracted with an additional 5 mL of hexanes solvent. The organic solution was dried under reduced pressure to yield the product as a pale-yellow liquid. Isolated 1.97 g, (6.74 mmol) for an 89.9% yield. ¹H-, ¹³C-, and ¹¹⁹Sn-NMR were recorded on a concentrated C₆D₆ solution of the product to confirm the target molecule has been synthesized in >93% initial purity. FIG. 13 displays ¹¹⁹Sn-NMR of isopropenylSn(NMe₂)₃ synthesized from isopropenylSnCl₃ in a hexanes solvent recorded in C₆D₆.

For ¹¹⁹Sn-NMR of isopropenylSn(NMe₂)₃ recorded in CDs. ¹¹⁹Sn{¹H}-NMR (149 MHz, CDe, 298K): −87.4 ppm (1.0%), −99.6 ppm (93.1%), −119.0 ppm (5.9%).

Aspects

Various Aspects are described below. It is to be understood that any one or more of the features recited in the following Aspect(s) can be combined with any one or more other Aspect(s).

Aspect 1. A method comprising: obtaining an alkyltintrihalide; obtaining a solvent, a solution, or any combination thereof; and contacting the alkyltintrihalide with the solvent, the solution, or any combination thereof so as to form an alkyltintrihalide adduct.

Aspect 2. The method of Aspect 1, wherein the method does not comprise forming an alkyltintrihalide-amine adduct.

Aspect 3. The method of Aspect 1 or 2, wherein the alkyltintrihalide-amine adduct is an alkyltintrihalide-(HNMe₂) adduct.

Aspect 4. The method as in any of the preceding Aspects, wherein the alkyltintrihalide is a compound of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

Aspect 5. The method of Aspect 4, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

Aspect 6. The method of Aspect 4, wherein R is a vinyl, an allyl, a propynyl, a propenyl, or any isomer thereof.

Aspect 7. The method as in any of the preceding Aspects, wherein the solvent comprises at least one of tetrahydrofuran (THF), dimethoxyethane (DME), or any combination thereof.

Aspect 8. The method as in any one of the Aspects 1-6, wherein the solution comprises at least one of hexane, pentane, toluene, or any combination thereof.

Aspect 9. The method as in of any of the preceding Aspects, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

Aspect 10. The method as in of any of the preceding Aspects, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

Aspect 11. A method comprising: obtaining an alkyltintrihalide adduct; obtaining a lithium dialkylamide; and contacting the alkyltintrihalide adduct and the lithium dialkylamide so as to form a tris(dialkylamido)alkyl tin product.

Aspect 12. The method of Aspect 11, wherein the method does not comprise forming an alkyltintrihalide-amine adduct.

Aspect 13. The method of Aspect 12, wherein the alkyltintrihalide-amine adduct is an alkyltintrihalide-(HNMe₂) adduct.

Aspect 14. The method as in of any of the preceding Aspects, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

Aspect 15. The method of Aspect 14, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

Aspect 16. The method of Aspect 14, wherein R is a vinyl, an allyl, a propynyl, a propenyl, or any isomer thereof.

Aspect 17. The method of Aspect 14, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, hexane, methanol, methyl t-butyl ether, methylene chloride, N-methyl-2-pyrrolidinone, pentane, petroleum ether, propanol, pyridine, tetrahydrofuran, toluene, triethylamine, water, xylene, any isomer thereof, or any combination thereof.

Aspect 18. The method as in of any of the preceding Aspects, wherein the lithium dialkylamide is a compound of the formula: LiN(R¹)₂, wherein: R¹ comprises a C₁-C₃ alkyl.

Aspect 19. The method as in of any of the preceding Aspects, wherein the tris(dialkylamido)alkyl tin product is a compound of the formula:

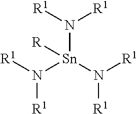

wherein R is a substituted C₁-C₅ alkyl, an unsubstituted C₁-C₅ alkyl, a substituted C₁-C₅ alkenyl, or an unsubstituted C₁-C₅ alkenyl; and wherein each R¹ is independently a C₁-C₃ alkyl.

Aspect 20. A method comprising: obtaining an alkyltintrihalide; obtaining a solvent, a solution, or any combination thereof; and contacting the alkyltintrihalide with the solvent, the solution, or any combination thereof so as to form an alkyltintrihalide adduct; obtaining a lithium dialkylamide; and contacting the alkyltintrihalide adduct and the lithium dialkylamide so as to form a tris(dialkylamido)alkyl tin product.

Aspect 21. The method of Aspect 20, wherein the method does not comprise forming an alkyltintrihalide-amine adduct.

Aspect 22. The method of Aspect 21, wherein the alkyltintrihalide-amine adduct is an alkyltintrihalide-(HNMe₂) adduct.

Aspect 23. The method as in of any of the preceding Aspects, wherein the alkyltintrihalide is a compound of the formula: RSnX₃, wherein: R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

Aspect 24. The method of Aspect 23, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

Aspect 25. The method of Aspect 23, wherein R is a vinyl, an allyl, a propynyl, a propenyl, or any isomer thereof.

Aspect 26. The method as in of any of the preceding Aspects, wherein the solvent comprises at least one of tetrahydrofuran (THF), dimethoxyethane (DME), or any combination thereof.

Aspect 27. The method as in of any of the preceding Aspects, wherein the solution comprises at least one of hexane, pentane, toluene, or any combination thereof.

Aspect 28. The method as in of any of the preceding Aspects, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

Aspect 29. The method as in of any of the preceding Aspects, wherein the alkyltintrihalide adduct is a compound of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

Aspect 30. The method as in of any of the preceding Aspects, wherein the lithium dialkylamide is a compound of the formula: LiN(R¹)₂, wherein: R¹ comprises a C₁-C₃ alkyl.

Aspect 31. The method as in of any of the preceding Aspects, wherein the tris(dialkylamido)alkyl tin product is a compound of the formula:

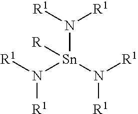

wherein R is a substituted C₁-C₅ alkyl, an unsubstituted C₁-C₅ alkyl, a substituted C₁-C₅ alkenyl, or an unsubstituted C₁-C₅ alkenyl; wherein each R¹ is independently a C₁-C₃ alkyl.

Aspect 32. A composition comprising: an alkyltintrihalide adduct of the formula: RSnX₃·(solv)ₙ, wherein: R is a substituted C₁-C₅ alkyl, an unsubstituted C₁-C₅ alkyl, a substituted C₁-C₅ alkenyl, or an unsubstituted C₁-C₅ alkenyl; X is Cl, Br, or I; solv is a solvent; and n is at least 1.

Aspect 33. The composition of Aspect 32, wherein the solvent comprises at least one of tetrahydrofuran (THF), dimethoxyethane (DME), hexane, or any combination thereof.

Aspect 34. The composition of Aspect 32, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, hexane, methanol, methyl t-butyl ether, methylene chloride, N-methyl-2-pyrrolidinone, pentane, petroleum ether, propanol, pyridine, tetrahydrofuran, toluene, triethylamine, water, xylene, any isomer thereof, or any combination thereof.

Aspect 35. A composition comprising: a tris(dialkylamido)alkyl tin product of the formula:

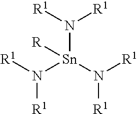

wherein R is a substituted C₁-C₅ alkyl, a substituted C₁-C₅ alkenyl, or an unsubstituted C₁-C₅ alkenyl; wherein each R¹ is independently a C₁-C₃ alkyl.

Aspect 36. The composition of Aspect 35, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂. In some aspects, the techniques described herein relate to a method, wherein R is an alkoxy.

Aspect 37. The composition of Aspect 35, wherein R is a vinyl, an allyl, a propynyl, a propenyl, or any isomer thereof.

Aspect 38. A composition comprising: a reaction product of an alkyltintrihalide adduct and a lithium dialkylamide, wherein the reaction product comprising a compound of the formula:

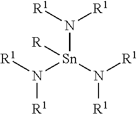

wherein R is a substituted C₁-C₅ alkyl, an unsubstituted C₁-C₅ alkyl, a substituted C₁-C₅ alkenyl, or an unsubstituted C₁-C₅ alkenyl; wherein each R¹ is independently a C₁-C₅ alkyl.

Aspect 39. The composition of Aspect 38, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

Aspect 40. The composition of Aspect 38, wherein R is a vinyl, an allyl, a propynyl, a propenyl, or any isomer thereof.

Aspect 41. A composition comprising:
an atomic layer deposition precursor comprising an alkyltintrihalide of the formula:

RSnX₃,
wherein:
   R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

Aspect 42. The composition of Aspect 41, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

Aspect 43. The composition of Aspect 41, wherein R is a vinyl, an allyl, a propynyl, a propenyl, or any isomer thereof.

Aspect 44. A composition comprising:
a chemical vapor deposition precursor comprising an alkyltintrihalide of the formula:

RSnX₃,
wherein:
   R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

Aspect 45. The composition of Aspect 44, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

Aspect 46. The composition of Aspect 44, wherein R is a vinyl, an allyl, a propynyl, a propenyl, or any isomer thereof.

Aspect 47. A composition comprising:
a compound of the formula:

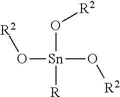

wherein:
   R is a substituted C₁-C₅ alkyl, an unsubstituted C₁-C₅ alkyl, a substituted C₁-C₅ alkenyl, or an unsubstituted C₁-C₅ alkenyl; R² is independently a substituted C₁-C₄ alkyl or an unsubstituted C₁-C₄ alkyl,
      wherein the substituted C₁-C₄ alkyl comprises a fluorine-containing substituent.

Aspect 48. The composition of Aspect 47, wherein the C₁-C₄ alkyl of R² is methyl, ethyl, n-propyl, iso-propyl, n-butyl, sec-butyl, or tert-butyl.

Aspect 49. The composition of Aspect 47, wherein the fluorine-containing substituent comprises —CH₂CF₃, —CH(CF₃)₂, or —(CHₐ)ₙ(CH_{b}F_{c})ₘ,
wherein:
   a=0 to 3;
   b=0 to 2;
   c=1 to 3;
   n=0 to 3;
   m=1 to 4.

Aspect 50. The composition of Aspect 49, wherein —(CHₐ)ₙ(CH_{b}F_{c})ₘ is —CH₂F, —CH₂CH₂F, —CF₃, or —CF₂CF₃.

Aspect 51. The composition of Aspect 47, wherein R² is a saturated alkyl or an unsaturated alkyl.

Aspect 52. The composition of Aspect 47, wherein OR² is —OCH₂C≡CH or —OCH═CH₂.

Aspect 53. A composition comprising:
a compound of the formula:

RSn(OR²)₃
wherein:
   R is a substituted C₁-C₅ alkyl, an unsubstituted C₁-C₅ alkyl, a substituted C₁-C₅ alkenyl, or an unsubstituted C₁-C₅ alkenyl; R² is independently a substituted C₁-C₄ alkyl or an unsubstituted C₁-C₄ alkyl,
      wherein the substituted C₁-C₄ alkyl comprises a fluorine-containing substituent.

Aspect 54. The composition of Aspect 53, wherein the C₁-C₄ alkyl of R² is methyl, ethyl, n-propyl, iso-propyl, n-butyl, sec-butyl, or tert-butyl.

Aspect 55. The composition of Aspect 53, wherein the fluorine-containing substituent comprises —CH₂CF₃, —CH(CF₃)₂, or —(CHₐ)ₙ(CH_{b}F_{c})ₘ,
wherein:
   a=0 to 3;
   b=0 to 2;
   c=1 to 3;
   n=0 to 3;
   m=1 to 4.

Aspect 56. The composition of Aspect 55, wherein —(CHₐ)ₙ(CH_{b}F_{c})ₘ is —CH₂F, —CH₂CH₂F, —CF₃, or —CF₂CF₃.

Aspect 57. The composition of Aspect 53, wherein R² is a saturated alkyl or an unsaturated alkyl.

Aspect 58. The composition of Aspect 53, wherein OR² is —OCH₂C≡CH or —OCH═CH₂.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A method comprising:
obtaining an alkyltintrihalide;
obtaining a solvent, a solution, or any combination thereof; and
contacting the alkyltintrihalide with the solvent, the solution, or any combination thereof so as to form an alkyltintrihalide adduct.

2. The method of claim 1, wherein the method does not comprise forming an alkyltintrihalide-amine adduct.

3. The method of claim 2, wherein the alkyltintrihalide-amine adduct is an alkyltintrihalide-(HNMe₂) adduct.

4. The method of claim 1, wherein the alkyltintrihalide is a compound of the formula:
RSnX₃,
wherein:
R is a substituted C₁-C₅ alkyl, an unsubstituted C₁-C₅ alkyl, a substituted C₁-C₅ alkenyl, or an unsubstituted C₁-C₅ alkenyl; and X is Cl, Br, or I.

5. The method of claim 4, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

6. The method of claim 4, wherein R is a vinyl, an allyl, a propynyl, a propenyl, or any isomer thereof.

7. The method of claim 1, wherein the solvent comprises at least one of tetrahydrofuran (THF), dimethoxyethane (DME), or any combination thereof.

8. The method of claim 1, wherein the solution comprises at least one of hexane, pentane, toluene, or any combination thereof.

9. The method of claim 1, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, methanol, methyl t-butyl ether, methylene chloride, N-methyl-2-pyrrolidinone, petroleum ether, propanol, pyridine, tetrahydrofuran, triethylamine, water, xylene, any isomer thereof, or any combination thereof.

10. The method of claim 1, wherein the alkyltintrihalide adduct is a compound of the formula:
RSnX₃·(solv)ₙ,
wherein:
R is a substituted C₁-C₅ alkyl, an unsubstituted C₁-C₅ alkyl, a substituted C₁-C₅ alkenyl, or an unsubstituted C₁-C₅ alkenyl;
X is Cl, Br, or I;
solv is a solvent; and
n is at least 1.

11. A method comprising:
obtaining an alkyltintrihalide adduct;
obtaining a lithium dialkylamide; and
contacting the alkyltintrihalide adduct and the lithium dialkylamide so as to form a tris(dialkylamido)alkyl tin product.

12. The method of claim 11, wherein the method does not comprise forming an alkyltintrihalide-amine adduct.

13. The method of claim 12, wherein the alkyltintrihalide-amine adduct is an alkyltintrihalide-(HNMe₂) adduct.

14. The method of claim 11, wherein the alkyltintrihalide adduct is a compound of the formula:
RSnX₃·(solv)ₙ,
wherein:
R is a substituted C₁-C₅ alkyl, an unsubstituted C₁-C₅ alkyl, a substituted C₁-C₅ alkenyl, or an unsubstituted C₁-C₅ alkenyl;
X is Cl, Br, or I;
solv is a solvent; and
n is at least 1.

15. The method of claim 14, wherein R is a methyl, an ethyl, a n-propyl, a cyclopropyl, an isopropyl, an n-butyl, a t-butyl, a sec-butyl, an n-pentyl, an isopentyl, a sec-pentyl, CF₃CH₂, CF₂HCH₂, CFH₂CH₂, or CFH₂.

16. The method of claim 14, wherein R is a vinyl, an allyl, a propynyl, a propenyl, or any isomer thereof.

17. The method of claim 14, wherein the solvent comprises at least one of acetic acid, acetone, acetonitrile, benzene, butanol, butanone, t-butyl alcohol, carbon tetrachloride, chlorobenzene, chloroform, cyclohexane, 1,2-dichloroethane, diethylene glycol, diethyl ether, diethylene glycol dimethyl ether, dimethoxyethane, dimethylformamide, dimethyl sulfoxide, 1,4-dioxane, ethanol, ethyl acetate, ethylene glycol, glycerin, heptane, hexane, methanol, methyl t-butyl ether, methylene chloride, N-methyl-2-pyrrolidinone, pentane, petroleum ether, propanol, pyridine, tetrahydrofuran, toluene, triethylamine, water, xylene, any isomer thereof, or any combination thereof.

18. The method of claim 11, wherein the lithium dialkylamide is a compound of the formula:
LiN(R¹)₂,
wherein:
R¹ comprises a C₁-C₃ alkyl.

19. The method of claim 11, wherein the tris(dialkylamido)alkyl tin product is a compound of the formula: 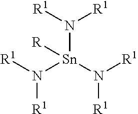
wherein R is a substituted C₁-C₅ alkyl, an unsubstituted C₁-C₅ alkyl, a substituted C₁-C₅ alkenyl, or an unsubstituted C₁-C₅ alkenyl; and wherein each R¹ is independently a C₁-C₃ alkyl.

20. A method comprising:
obtaining an alkyltintrihalide;
obtaining a solvent, a solution, or any combination thereof; and
contacting the alkyltintrihalide with the solvent, the solution, or any combination thereof so as to form an alkyltintrihalide adduct;
obtaining a lithium dialkylamide; and
contacting the alkyltintrihalide adduct and the lithium dialkylamide so as to form a tris(dialkylamido)alkyl tin product.
